(19) United States
(12) Patent Application Publication
(10) Pub. No.: US 2026/0363675 A1
(43) Pub. Date: Nov. 19, 2026

(54) Toilet-Attachable Solid Waste Collection Device

(71) Applicants: Joseph Vidricko, Joliet, IL (US); James Sossong, Plainfield, IL (US)

(72) Inventors: Joseph Vidricko, Joliet, IL (US); James Sossong, Plainfield, IL (US)

(21) Appl. No.: 19/238,905

(22) Filed: Jun. 16, 2025

Related U.S. Application Data
(60) Provisional application No. 63/804,748, filed on May 13, 2025.

(51) Int. Cl. A61B 10/00

(52) U.S. Cl. CPC A61B 10/0038

(57) ABSTRACT

A toilet-attachable solid waste collection device is provided that is configured for removable or semi-permanent installation within a conventional toilet bowl via an attachment interface. The body may be various shapes to ergonomically accommodate user anatomy and toilet geometries, and may be made from medical-grade, non-stick materials for ease of sanitation. A removable filter liner is inserted into the body that may be comprised of multi-layered structure with a coarse mesh for large particulates, a finer mesh for smaller waste elements, and an odor-control layer infused with adsorptive compounds such as activated carbon. Below the liner, the body is comprised of perforations that allow for the controlled passage of liquid waste into the toilet bowl. In various embodiments, the liner may be reusable or disposable.

### CROSS-REFERENCE TO RELATED APPLICATION

The present application claims priority to, and the benefit of, U.S. Provisional Application No. 63/804,748, which was filed on May 13, 2025, and is incorporated herein by reference in its entirety.

### FIELD OF THE INVENTION

The present invention relates generally to the field of waste collection. More specifically, the present invention relates to a solid waste collection assembly that can be attached to a toilet to collect solid waste prior to flushing. Accordingly, the present disclosure makes specific reference thereto. Nonetheless, it is to be appreciated that aspects of the present invention are also equally applicable to other like applications, devices, and methods of manufacture.

### BACKGROUND

Toilet plumbing is optimized for moderate waste volume, and any deviation from this can result in partial or complete clogs of the system. Such clogs can lead to unsanitary conditions, particularly when users attempt manual removal or plunging, which exposes them to potentially hazardous biological material. Furthermore, many public and residential users may lack the tools, physical ability, or willingness to address these clogs, thereby exacerbating hygiene and maintenance challenges. The inconvenience and risk of exposure contribute to user dissatisfaction and increased maintenance costs. Additionally, toilets with recurring clogging issues may result in higher water usage due to repeated flushing attempts. These limitations collectively indicate a systemic flaw in conventional waste disposal design and underscore the necessity for a supplementary solution. Additionally, there are individuals with medical conditions or those undergoing diagnostic procedures who must collect stool samples at home under hygienic conditions. Traditional methods of stool sample collection can be difficult, unsanitary, and imprecise, especially for elderly or mobility-impaired users. The lack of specialized, toilet-compatible tools for this purpose contributes to user discomfort and increases the likelihood of cross-contamination. These issues highlight the broader limitations of conventional toilet designs, which do not account for the specialized handling, separation, or sampling of solid waste.

Therefore, there exists a long-felt need in the art for a toilet-attachable solid waste collection device that prevents the accumulation of solid waste and toilet paper from obstructing toilet function. There also exists a long-felt need in the art for a toilet-attachable solid waste collection device that enables sanitary removal and disposal of human waste without requiring manual interaction with clogged plumbing. Moreover, there exists a long-felt need in the art for a toilet-attachable solid waste collection device that provides a hygienic and user-friendly means for stool sample collection for diagnostic and medical purposes.

The subject matter disclosed and claimed herein, in one embodiment thereof, comprises a toilet-attachable solid waste collection device. The device is comprised of a solid waste collection assembly configured for removable or semi-permanent installation within a conventional toilet bowl via an attachment interface. The body may be various shapes to ergonomically accommodate user anatomy and toilet geometries, and may be made from medical-grade, non-stick materials for ease of sanitation. A removable filter liner is inserted into the body that may be comprised of a multi-layered structure with a coarse mesh for large particulates, a finer mesh for smaller waste elements, and an odor-control layer infused with adsorptive compounds such as activated carbon. Below the liner, the body is comprised of perforations that allow for the controlled passage of liquid waste into the toilet bowl. In various embodiments, the liner may be reusable or disposable.

In this manner, the toilet-attachable solid waste collection device of the present invention accomplishes all the foregoing objectives and provides a device that allows for pre-flush separation of solid and liquid waste, thereby reducing the incidence of clogs. The multi-layered filter liner provides a sanitary and user-friendly interface for waste capture and disposal, while the handle-equipped design enhances hygiene and ease of use. Importantly, the design includes features that enable accurate and sanitary stool sample collection, supporting diagnostic and medical needs. Furthermore, compatibility with various toilet designs and the inclusion of tool-free mounting systems ensure broad usability. Thus, the device provides a comprehensive, sanitary solution to the problems of waste-related plumbing disruptions and medical waste handling.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a toilet-attachable solid waste collection device. The device is comprised of a solid waste collection assembly configured for removable or semi-permanent installation onto a conventional toilet seat. The assembly is suspended within a toilet bowl and does not interfere with standard toilet functions such as flushing, seating, or lid closure.

The device is comprised of a collection body configured to collect solid waste. The collection body may be shaped in various ergonomic forms to accommodate different toilets or user preferences and may be constructed from sanitary, non-stick materials such as PTFE-coated polypropylene or silicone-infused thermoplastics. The internal surfaces of the collection body may be contoured or inclined to direct waste toward a centralized area. The collection body may further be comprised of at least one handle to assist with removal.

The body may include perforations located below the filter liner to permit liquid waste drainage into the toilet bowl. These perforations may vary in configuration to optimize flow, prevent clogging, and minimize splash-back. The internal surface of the collection body may be treated with hydrophobic, oleophobic, or antimicrobial coatings applied by spray, plasma, or thermal methods to reduce residue buildup and microbial growth.

The body may receive a filter liner designed to reduce splatter and enable waste separation and odor control. The filter liner may be comprised of multiple layers, including an upper mesh for capturing large particulates, a secondary finer mesh for smaller particulates, and a tertiary odor-control layer infused with adsorptive compounds such as activated carbon or zeolite. The mesh layers may be constructed from synthetic fibers or stainless steel based on reusability requirements.

The filter liner may be detachable and configured for either single-use or reusable applications. Reusable liners may be comprised of washable, autoclavable materials, while disposable versions may use biodegradable or compostable materials. At least one handle may be included on the liner for hygienic removal.

The device may be attached to a toilet seat using an attachment interface comprised of mechanical or adhesive components such as suction cups, clips, brackets, magnets, or clamps. The attachment interface is preferably tool-free to allow easy installation and removal. In one embodiment, the attachment interface may be comprised of flexible mounting brackets with a lip designed to fit beneath a toilet seat rim, securing the device without altering the seat's structure.

A method of using the device involves providing the collection body, filter liner, and attachment interface, then securing the device to a toilet seat so the collection body is suspended inside the bowl. The filter liner is inserted into the collection body before use. During use, solid waste is collected by the filter while liquid waste drains through the perforations. After use, the liner is removed for disposal or cleaning, followed by detachment and cleaning of the collection body.

Accordingly, the toilet-attachable solid waste collection device of the present invention is particularly advantageous as it provides a device that allows for pre-flush separation of solid and liquid waste, thereby reducing the incidence of clogs. The multi-layered filter liner provides a sanitary and user-friendly interface for waste capture and disposal, while the handle-equipped design enhances hygiene and ease of use. Importantly, the design includes features that enable accurate and sanitary stool sample collection, supporting diagnostic and medical needs. Furthermore, compatibility with various toilet designs and the inclusion of tool-free mounting systems ensure broad usability. As a result, the device provides a comprehensive, sanitary solution to the problems of waste-related plumbing disruptions and medical waste handling that overcomes the limitations of existing toilets known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a toilet-attachable solid waste collection device of the present invention with a body, a liner, and a liner within the body in accordance with the disclosed architecture;

FIG. 2 illustrates a perspective view of one potential embodiment of a toilet-attachable solid waste collection device of the present invention while attached to and within a toilet in accordance with the disclosed architecture;

FIG. 3 illustrates a side cross-sectional view of a liner of one potential embodiment of a toilet-attachable solid waste collection device of the present invention in accordance with the disclosed architecture; and

FIG. 4 illustrates a flowchart of a method of using one potential embodiment of a toilet-attachable solid waste collection device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a toilet-attachable solid waste collection device that prevents the accumulation of solid waste and toilet paper from obstructing toilet function. There also exists a long-felt need in the art for a toilet-attachable solid waste collection device that enables sanitary removal and disposal of human waste without requiring manual interaction with clogged plumbing. Moreover, there exists a long-felt need in the art for a toilet-attachable solid waste collection device that provides a hygienic and user-friendly means for stool sample collection for diagnostic and medical purposes.

The present invention, in one exemplary embodiment, is comprised of a toilet-attachable solid waste collection device designed for either removable or semi-permanent installation onto a standard toilet seat is provided. The assembly is suspended within the toilet bowl in a manner that maintains compatibility with standard toilet operations, including flushing, seating, and lid closure.

The device includes a body for solid waste, formed in a variety of ergonomic shapes to suit different toilet models and user preferences. This component is fabricated from sanitary, non-stick materials such as PTFE-coated polypropylene or silicone-infused thermoplastics. The interior of the collection body may feature contoured or inclined surfaces to direct waste toward a centralized area. At least one handle may be integrated to facilitate removal.

Perforations positioned beneath the filter liner within the collection body allow liquid waste to drain into the toilet bowl. These perforations may vary in form to optimize flow rate, reduce splash-back, and minimize clogging. The internal surface may be treated with hydrophobic, oleophobic, or antimicrobial coatings applied through spray, plasma, or thermal deposition to prevent residue accumulation and microbial growth.

A filter liner, which is designed to control splatter and manage waste separation and odor, may be received within the collection body. The liner may include multiple layers: an upper mesh layer for large particulates, a secondary mesh for finer material, and a tertiary odor-control layer infused with compounds such as activated carbon or zeolite. Material choices for the mesh may include synthetic fibers or stainless steel, based on reusability.

The filter liner is detachable and may be intended for single-use or reuse. Reusable versions may be constructed from washable, autoclavable materials, while disposable variants may utilize biodegradable or compostable compositions. At least one handle may be included to facilitate sanitary removal.

The device may be affixed to a toilet seat using an attachment interface comprised of mechanical or adhesive mechanisms such as suction cups, clips, brackets, magnets, or clamps. This interface is designed for tool-free installation and removal. In one embodiment, the interface may feature flexible mounting brackets with a lip configured to secure beneath the toilet seat rim without deforming the seat structure.

A method of use involves supplying the collection body, filter liner, and attachment interface, followed by securing the device to the toilet seat to suspend the body within the bowl. The filter liner is inserted prior to use. Solid waste is captured by the filter while liquid waste drains through the perforations. After use, the liner is removed for disposal or cleaning, and the collection body is detached and cleaned.

As a result, the device provides a pre-flush separation system for solid and liquid waste, thereby mitigating plumbing clogs. The multi-layered liner offers a sanitary and convenient interface for waste handling, with an integrated handle system to enhance hygiene. The device further supports accurate and sanitary stool sample collection for diagnostic purposes. In addition, the device has broad compatibility with toilet designs and the use of tool-free attachment systems. Therefore, the device delivers a sanitary and effective solution to issues related to plumbing blockages and medical waste management, addressing limitations present in existing toilet systems.

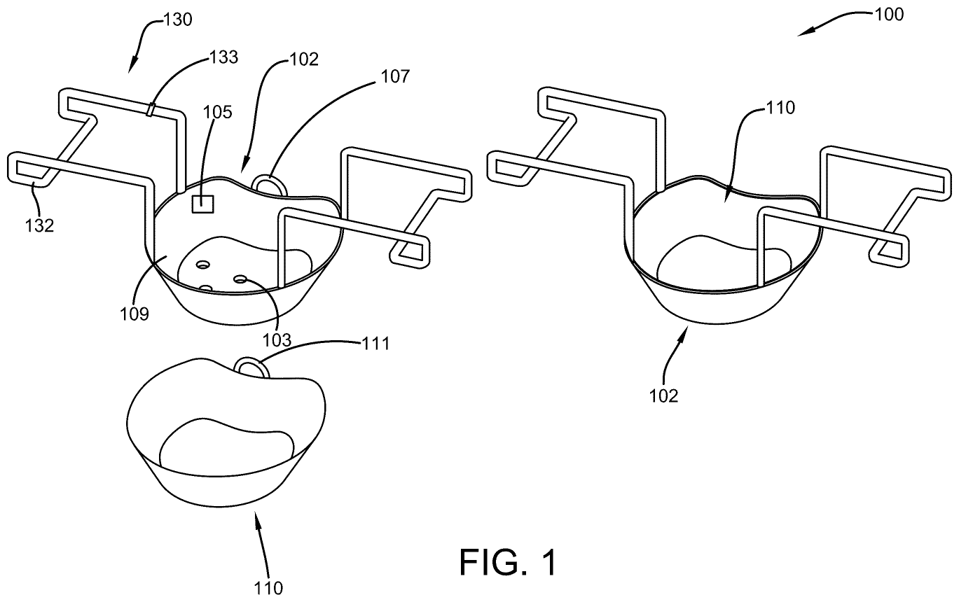

Referring initially to the drawings, FIG. 1 illustrates a perspective view of one potential embodiment of a toilet-attachable solid waste collection device 100 of the present invention with a body 102, a liner 110, and a liner 110 within the body 102 in accordance with the disclosed architecture. The device 100 may be comprised of a solid waste collection assembly designed for removable or semi-permanent installation onto a conventional toilet seat such that the assembly is suspended within a toilet bowl without interfering with typical toilet operations, including flushing, seating, and lid closure.

The device 100 may be comprised of a body 102 used for collecting solid waste. The body 102 may be any shape, such as, but not limited to, oval, elliptical, funnel-shaped, conical, or irregular ergonomic shapes, and may vary in depth, width, and contouring to accommodate different toilet models or user anatomical preferences. The collection body 102 may be constructed from materials such as, but not limited to, medical-grade, non-stick polymers, including PTFE-coated polypropylene, silicone-infused thermoplastics, or similar materials selected for ease of cleaning, chemical resistance, and sanitary compatibility. In some configurations, the internal surfaces of the collection body 102 may be contoured, inclined, or channeled to direct deposited waste materials toward a centralized area of the body 102. In one embodiment, the body 102 may be comprised of at least one handle 107 of any size, shape, and configuration to facilitate removal.

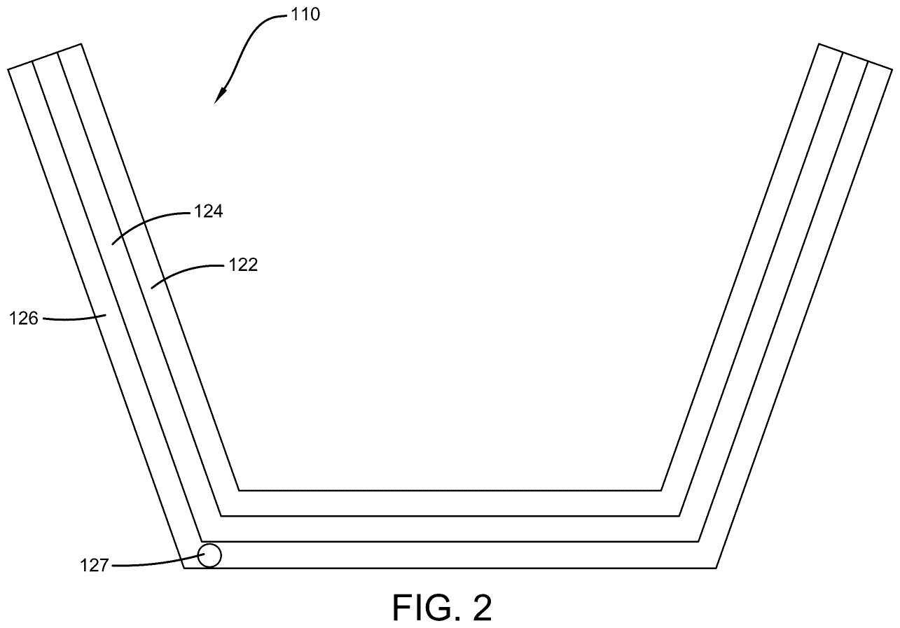

In one embodiment, the body 102 may receive at least one filter liner 110 configured to minimize splatter during use, as seen in FIG. 1. In one embodiment, the filter liner 110 may be comprised of a multi-layered structure for sequential waste separation and odor control, as seen in FIG. 2. In one embodiment, the liner 110 may be comprised of an upper mesh layer 122 configured to capture larger solid waste particulates. The mesh 122 may be comprised of durable synthetic fibers or medical-grade stainless steel, depending on reusability requirements. A secondary mesh layer 124 may be positioned beneath the upper mesh 122 and may be comprised of finer filtration material to trap smaller particulate matter. A tertiary odor-control layer 126 may be positioned beneath the finer mesh 124, where the odor-control layer 126 may be comprised of a non-woven fiber mat infused with adsorptive compounds 127, such as, but not limited to, activated carbon, zeolite crystals, or molecular sieves for odor mitigation.

The filter liner 110 may be detachable from the collection body 102 (as seen in FIG. 1) and may be configured for either single-use or multiple-use applications. In reusable embodiments, the liner 110 may be constructed from washable and autoclavable synthetic fiber composites. In disposable embodiments, the liner 110 may be fabricated from biodegradable polymer blends, compostable bio-based materials, and/or odor eliminating/mitigating materials that may be comprised of adsorptive compounds 127. In one embodiment, the liner 110 may be comprised of at least one handle 111 for removal, where the handle 111 may include reinforced tabs or loop structures for hygienic retrieval.

The collection body 102 may include one or more perforations 103 (as seen in FIG. 1) strategically positioned below the filter liner 110. The perforations 103 may be configured to permit the drainage of liquid waste into the underlying toilet bowl. The size, shape, and distribution pattern of the perforations 103 may vary across different embodiments to optimize flow rate, prevent clogging, and minimize splash-back.

In one embodiment, the internal surface 109 of the collection body 102 may be treated with one or more surface treatments 105, such as, but not limited to, hydrophobic, oleophobic, or antimicrobial coatings. These treatments 105 may be applied via spray coating, plasma treatment, or thermal deposition methods to reduce residue buildup and inhibit microbial colonization.

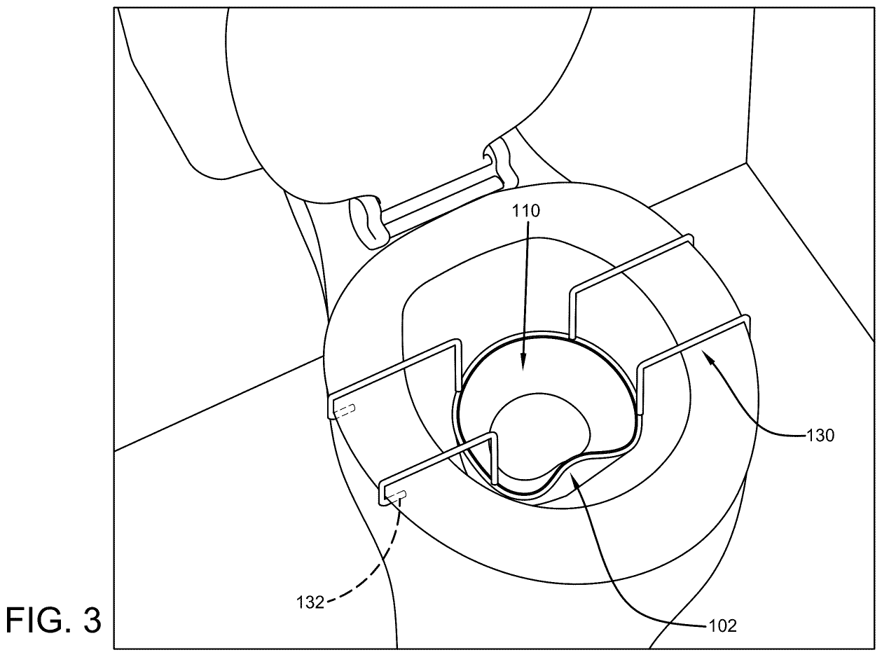

The device 100 may be secured to a toilet seat via an attachment interface 130, as seen in FIG. 3. The attachment interface 130 may be comprised of mechanical or adhesive engagement elements, such as, but not limited to, suction cups, snap-fit fasteners, adjustable clips, flexible mounting brackets, adhesives, magnets, or clamping systems. The attachment interface 130 is preferably designed for tool-free installation and removal, promoting ease of use and hygienic handling.

In one preferred embodiment, the attachment interface 130 is comprised of flexible mounting brackets that include a lip 132, as seen in FIG. 1 and FIG. 3. The lip 132 may be configured to fit around and underneath a standard toilet seat rim, anchoring the device 100 securely in place without deforming the toilet seat structure.

In one additional or alternative embodiment, the attachment interface 130 may be telescopic and may be comprised of at least one locking mechanism 133. The locking mechanism 133 may include, but is not limited to, rotary cams, spring-loaded detents, ratchet-and-groove configurations, threaded couplings, etc. This configuration enables the overall width of the device 100 to be adjusted, allowing compatibility with a range of toilet seat sizes and designs.

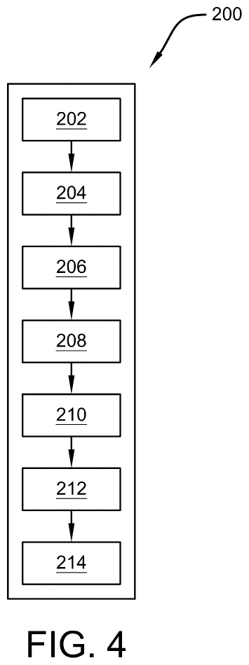

The present invention is also comprised of a method of using 200 the device 100, as seen in FIG. 4. First, a device 100 is provided comprised of a body 102, a filter liner 110, an attachment interface 130, and optionally at least one handle 107 and at least one surface treatment 105 [Step 202]. Then, the attachment interface 130 can be attached/secured onto a conventional toilet seat such that the collection body 102 is suspended within the toilet bowl without interfering with normal toilet operations [Step 204]. Next, the filter liner 110 can be inserted into the collection body 102 [Step 206]. Then, the user may utilize the toilet in a typical manner, during which solid waste is directed onto the filter 110 and sequentially captured and filtered through the underlying filter 110 layers while liquid waste passes through the perforations 103 into the toilet bowl [Step 208]. Upon completion of use, the liner 110 may be removed from the body 102 (in one embodiment by gripping at least one handle 111 and lifting the liner out of the collection body 102) for disposal or cleaning, depending on whether the liner 110 is disposable or reusable [Step 210]. Next, the collection body 102 may be removed from the toilet seat by disengaging the attachment interface 130, such as by lifting flexible mounting brackets or unlocking any telescopic mechanisms 133 [Step 212]. Finally, the collection body 102 may be cleaned using appropriate sanitary procedures [Step 214].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “toilet-attachable solid waste collection device” and “device” are interchangeable and refer to the toilet-attachable solid waste collection device 100 of the present invention.

Notwithstanding the foregoing, the toilet-attachable solid waste collection device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the toilet-attachable solid waste collection device 100 as shown in the FIGS. are for illustrative purposes only, and that many other sizes and shapes of the toilet-attachable solid waste collection device 100 are well within the scope of the present disclosure. Although the dimensions of the toilet-attachable solid waste collection device 100 are important design parameters for user convenience, the toilet-attachable solid waste collection device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A toilet-attachable solid waste collection device comprising:
a body configured to collect a solid waste, the body comprised of a perforation;
a filter liner removably positioned within the body; and
an attachment interface configured to removably secure the body to a toilet seat.

2. The toilet-attachable solid waste collection device of claim 1, wherein the body is contoured.

3. The toilet-attachable solid waste collection device of claim 1, wherein the attachment interface is comprised of a flexible mounting bracket.

4. The toilet-attachable solid waste collection device of claim 3, wherein the flexible mounting bracket is comprised of a lip.

5. The toilet-attachable solid waste collection device of claim 1, wherein the attachment interface is comprised of a clip.

6. A toilet-attachable solid waste collection device comprising:
a body configured to collect a solid waste, the body comprised of a perforation;
a filter liner removably positioned within the body, the liner comprised of an odor-adsorptive compound; and
an attachment interface configured to removably secure the body to a toilet seat.

7. The toilet-attachable solid waste collection device of claim 6, wherein the filter is comprised of an upper mesh layer.

8. The toilet-attachable solid waste collection device of claim 6, wherein the filter is comprised of a secondary mesh layer.

9. The toilet-attachable solid waste collection device of claim 6, wherein the odor-adsorptive compound is comprised of an activated carbon.

10. The toilet-attachable solid waste collection device of claim 6, wherein the odor-adsorptive compound is comprised of a zeolite crystal.

11. The toilet-attachable solid waste collection device of claim 6, wherein the odor-adsorptive compound is comprised of a molecular sieve.

12. The toilet-attachable solid waste collection device of claim 6, wherein the body is comprised of a first handle.

13. The toilet-attachable solid waste collection device of claim 6, wherein the liner is comprised of a second handle.

14. The toilet-attachable solid waste collection device of claim 6, wherein the attachment interface is comprised of a lip.

15. The toilet-attachable solid waste collection device of claim 6, wherein the attachment interface is telescopic.

16. The toilet-attachable solid waste collection device of claim 6, wherein the attachment interface is comprised of a locking mechanism.

17. The toilet-attachable solid waste collection device of claim 6, wherein the body is comprised of a hydrophobic coating.

18. The toilet-attachable solid waste collection device of claim 6, wherein the body is comprised of an oleophobic coating.

19. The toilet-attachable solid waste collection device of claim 6, wherein the body is comprised of an antimicrobial coating.

20. A method of using a toilet-attachable solid waste collection device, the method comprising the following steps:
providing a toilet-attachable solid waste collection device comprised of a body, a filter liner, and an attachment interface;
attaching the attachment interface to a toilet seat such that the body is suspended within a toilet bowl;
inserting the filter liner into the body;
using the toilet such a solid waste is captured by the filter liner and liquid waste passes through a perforation in the body into the toilet; and
removing the filter liner from the body for disposal or cleaning.
